(19) United States
(12) Patent Application Publication
(10) Pub. No.: US 2009/0117384 A1
(43) Pub. Date: May 7, 2009

(54) Titania Nanocavities and Method of Making

(75) Inventor: Weiqiang Han, Weiqiang, NY (US)

Correspondence Address: BROOKHAVEN SCIENCE ASSOCIATES/;BROOKHAVEN NATIONAL LABORATORY, BLDG. 185 - P.O. BOX 5000, UPTON, NY, 11973, US

(73) Assignee: Brookhaven Science Associates, LLC, Upton, NY (US)

(21) Appl. No.: 12/265,923

(22) Filed: Nov. 6, 2008

Related U.S. Application Data
(60) Provisional application No. 60/986,000, filed on Nov. 7, 2007.

(51) Int. Cl. G02B 5/22; B32B 5/16; A61K 8/29; C01G 23/04; H01L 31/00; H01M 4/58

(52) U.S. Cl. 428/402; 424/59; 423/608; 136/252; 429/231.95

(57) ABSTRACT

Disclosed herein are compositions of metal oxide nanoparticles having regular polyhedral nanocavities, where the metal oxide can be titania, and where the nanoparticles be nanorods. Also disclosed are titania nanoparticles with nanocavities that are doped with dopants. Methods of making metal oxide nanoparticles with nanocavities are also disclosed. Also disclosed are ultraviolet-blocking compositions including metal oxide nanoparticles with nanocavities, as well as methods of enhancing ultraviolet absorbance efficiency of an ultraviolet blocking composition. Additional uses of metal oxide nanoparticles with nanocavities include solar energy conversion systems and lithium-ion batteries.

### CROSS-REFERENCE TO RELATED APPLICATIONS

This application claims priority to U.S. Provisional Patent Application No. 60/986,000 filed on Nov. 7, 2007 under 35 U.S.C. § 119(e), the entirety of which is incorporated by reference as if fully set forth in this specification.

### STATEMENT OF GOVERNMENT LICENSE RIGHTS

The present invention was made with government support under contract number DE-AC02-98CH10886 awarded by the U.S. Department of Energy. The United States government has certain rights in the invention.

### BACKGROUND OF THE INVENTION

I. Field of the Invention

This invention relates generally to metal oxide nanoparticles having nanocavities. In particular, the present invention relates to titania nanorods having regular polyhedral nanocavities. This invention further relates to methods of making and using metal oxide nanoparticles with nanocavities.

II. Background of the Related Art

Titania, in particular anatase TiO₂, has been extensively used in photo-electrochemical systems, such as dye-sensitized TiO₂ electrodes for photovoltaics and solar cells, water splitting catalysts for hydrogen generation, and for the creation or degradation of specific compounds. Titania has also been extensively used in ultraviolet absorption products, such as sunscreen, cosmetics, and industrial coatings.

Several approaches have been attempted to improve the absorbance and photoreactivity of TiO₂, including doping (extension of its absorption wavelength into the visible region) and metal loading (for efficient electron-hole separation).

Theoretical calculations predict a significant enhancement of the optical absorbance in a thin silicon film with nanocavities, which indicates another route to increase the absorbance and photoreactivity of TiO₂ and other metal oxides. Nanocavities as described herein are isolated entities inside a solid, and are thus very different from nanopores, which (often being irregular and made from amorphous material) connect together and are open to the surface. Although nanoporous materials have been reported, there are relatively few reports of materials with nanocavities.

Nanocavities in solids have reportedly been made by irradiating a material with neutrons, gas ions, or heavy particles. Nanocavities in solids have also reportedly been made via a sol-gel method. Both of these methods are disadvantageous for the following reasons. In order to generate neutrons, gas ions, or heavy particles to irradiate solid materials, highly specialized, expensive, and sometimes dangerous equipment is required. Furthermore, these irradiation processes often damage the materials. The sol-gel method requires very expensive reagents and a complex synthetic procedure. These methods of producing nanocavities result in irregular nanocavities, both in size and in shape. Additionally, the bulk material used in these methods are often large-sized materials, and not nanoscale materials.

### SUMMARY OF THE INVENTION

In view of the above-mentioned disadvantages of the known methods, it as an objective of the present invention to provide a method for making a metal oxide nanoparticle with regular polyhedral nanocavities that is relatively simple, safe, and inexpensive to produce. It is a further goal of the present invention to provide metal oxide nanoparticles with regular polyhedral nanocavities which have an enhanced UV absorbance compared to analogous nanoparticles without the nanocavities.

In one embodiment, a composition comprising a plurality of metal oxide nanoparticles having regular polyhedral nanocavities is provided. The nanocavities are isolated from the surface of the nanoparticles. In a preferred embodiment, the metal oxide is TiO₂. In another preferred embodiment, the nanoparticles are nanorods. The nanorods can have diameters ranging from about 20 to about 150 nanometers. In yet another preferred embodiment, the nanocavities can have diameters ranging from about 5 to about 50 nanometers.

In a related embodiment, the metal oxide nanoparticles having regular polyhedral nanocavities are doped with a dopant. Dopants usable in this embodiment include, but are not limited to, lithium, sodium, potassium, boron, carbon, nitrogen, fluorine, aluminum, silicon, phosphorous, sulfur, calcium, scandium, vanadium, chromium, manganese, iron, cobalt, nickel, copper, and zinc. In preferred embodiments, the dopant is iron, nitrogen, or nickel.

Compositions of the invention have advantageously higher UV absorption when compared with analogous compositions without nanocavities. UV absorption in the content of the present disclosure means less than about 360 nm. Accordingly, in one embodiment, the compositions of the invention can be included in an ultraviolet-blocking composition. The ultraviolet-blocking compositions contemplated include, but are not limited to, sunscreen compositions, cosmetic compositions, and industrial coating compositions. In each of these embodiments, the ultraviolet-blocking compositions further include a cosmetically-acceptable carrier or an industrially acceptable carrier.

In a related embodiment, the compositions of the invention are used in a method for enhancing the efficiency of an ultraviolet-blocking composition. In such an embodiment, the composition of the invention comprising a plurality of metal oxide nanoparticles having regular polyhedral nanocavities is included in the ultraviolet-blocking composition.

In another embodiment, the compositions of the invention are used in a method for enhancing the efficiency of solar energy conversion. In such an embodiment, the composition of the invention comprising a plurality of metal oxide nanoparticles having regular polyhedral nanocavities is included in the solar energy conversion system.

In yet another embodiment, the compositions of the invention are used in a method for enhancing the lithium ion storage efficiency of lithium ion batteries. In such an embodiment, the composition of the invention comprising a plurality of metal oxide nanoparticles having regular polyhedral nanocavities is included in lithium ion batteries.

In an additional embodiment, the composition of the invention comprising a plurality of metal oxide nanoparticles having regular polyhedral nanocavities is prepared by a method comprising treating a powder of the oxide with an alkali solution in an autoclave at a temperature of from about 150° C. to about 190° C., washing the alkali-treated product with an acidic solution having from about 1% to about 15% by weight of a mineral acid, and heating the washed product at a temperature of from about 550° C. to about 750° C. in an atmosphere comprising oxygen or ammonia. In a preferred embodiment, the metal oxide is TiO₂.

In the above embodiment, the alkali treatment step is preferably accomplished using sodium hydroxide or potassium hydroxide solutions. The hydroxide is present in an amount ranging from about 5 molar to about 20 molar, preferably from about 10 molar to about 20 molar. The alkali treatment is preferably and advantageously performed at a temperature ranging from about 160° C. to about 180° C., and most preferably at about 170° C. The alkali treatment is performed for about 1 to about 7 days, preferably for about 2 to about 6 days, and more preferably for about 3 days.

In the above embodiment, the washing step is preferably accomplished using hydrochloric acid, nitric acid, sulfuric acid, or hydrofluoric acid, more preferably using nitric acid. The acid is preferably used in concentrations ranging from about 1% to about 15% by weight, more preferably from about 1.5% to about 10% by weight, and most preferably about 1.5% to about 3% by weight. The washing step is performed for about 4 to about 48 hours, preferably for about 4 to about 24 hours, and more preferably for about 12 to about 16 hours.

In the above embodiment, the washed product is heated at a temperature from about 550° C. to about 750° C., preferably from about 600° C. to about 700° C., and most preferably about 650° C. The washed product is heated under an atmosphere of oxygen or ammonia. Argon or nitrogen may also be added, so long as there is sufficient oxygen or ammonia to induce formation of nanocavities. The oxygen atmosphere can be air, flowing air, or pure oxygen. In a preferred embodiment, the heating is performed under oxygen. The heating is performed for about 1 to about 8 hours, preferably for about 2 to about 6 hours, and more preferably for about 2 to about 3 hours.

The above and other objectives and goals of the present invention will become more apparent from the following description and illustrative embodiments which are described in detail with reference to the accompanying drawings.

### BRIEF DESCRIPTION OF THE DRAWINGS

FIG. 1 shows the X-ray diffraction (XRD) spectrum of anatase TiO₂ nanorods with nanocavities prepared according to the present invention as described in Example 1.

FIG. 2 shows a low-magnification transmission electron microscopy (TEM) image of anatase TiO₂ nanorods with nanocavities prepared according to the present invention as described in Example 1.

FIG. 3 shows a high-magnification TEM image of anatase TiO₂ nanorods with nanocavities prepared according to the present invention as described in Example 1.

### DETAILED DESCRIPTION OF THE INVENTION

The preparation of titania nanorods with nanocavities in accordance with the present invention is accomplished by heat treatment of an intermediate hydrogen titanate. The intermediate hydrogen titanate is prepared by alkali treatment of TiO₂ particles inside an autoclave at temperatures ranging from about from about 150° C. to about 190° C. The hydrogen titanates usable in the preparation of compositions of the invention include, but are not limited to, H₂Ti₃O₇, H₂Ti₂O₅.H₂O, H₂Ti₅O₁₁.H₂O, H₂Ti₄O₉.19H₂O, (H₂O)_{0.25}Ti₄O₇(OH)₂, (H₂O)Ti₄O₇(OH)₂, H₂Ti₈O₁₇, H₂Ti₄O₁₇, and H₂Ti₄O₉.H₂O.

In a particular embodiment of the invention, H₂Ti₃O₇ is prepared as the intermediate hydrogen titanate by the NaOH treatment of TiO₂ particles inside an autoclave at a temperature range of 140 to 180° C. for 2-5 days, subsequently followed by acid washing. This procedure is similar to methods of making H₂Ti₃O₇ nanotubes, TiO₂ nanotubes, or other Ti—O-based ID nanostructures. In this embodiment, XRD results show that the intermediate product is monoclinic H₂Ti₃O₇, and TEM analyses show that most of the intermediate product are straight nanorods with diameters from 15 to 200 nm and lengths up to 10 μm. Very small amounts of short and thin H₂Ti₃O₇ nanotubes also exist in the intermediate product in this embodiment.

The heat treatment of the intermediate hydrogen titanates in accordance with the invention is performed at a temperature from about 550° C. to about 750° C. under an atmosphere of oxygen or ammonia. The oxygen atmosphere can be air or pure oxygen, flowing or static.

In the particular embodiment above, the intermediate hydrogen titanate nanorods (H₂Ti₃O₇) are heated in air at a temperature ranging from 600 to 720° C. XRD shows that the final product is single-phase anatase TiO₂. Overall TEM observations show that the nanorods have similar shape and size distributions as those of the intermediate H₂Ti₃O₇ nanorods. Most nanorods are single crystals. The nanorods contain numerous nanocavities evidenced at low-magnification TEM. The typical diameter of the nanocavities in this embodiment is about 10 nm.

When the nanorods of this embodiment are viewed along the principle directions, for example, the [100] direction, high magnification TEM shows the nanocavities to have a sharp polyhedral shape. The electron diffraction pattern taken from the whole nanorods was indexed as the (100)* pattern of anatase, indicating that the nanorods are still a single crystalline phase. Moreover, the nanocavities are rarely present near the edge of the nanorods, even when the nanorods are tilted ±30° along its axis direction. In this embodiment, one particular nanocavity was measured to be 8 nm deep and 12 nm wide. No elements other than Ti and O are present either in the nanorods or at the boundary of the nanocavity.

The nanocavities in this embodiment have a polyhedral shape. The boundaries between the nanocavities and the nanorod body are atomically sharp. The boundary planes are {01 1̅}, {100}, and {001}, which are all low-index planes of the anatase crystal and have the lowest surface formation energies.

While not intending to be limited in any way by theory, it is the inventor's belief that the nanocavities are formed according to an “anti-crystal growth” model, which parallels classic nucleation theory. The intermediate hydrogen titanate is made via a relatively low-temperature, wet-chemistry method and thus contains many defects, such as voids, which are favored places to start an “anti-nucleation” process during the heat treatment step. The free energy per unit volume favors the formation of the new phase (enlarged voids) while the surface energy (i.e., the surface energy of surrounded cogrowth TiO₂ crystal) disfavors the formation of the enlarged voids. In an O₂ background pressure, oxygen-deficient surfaces of TiO₂ react with electronegative O₂ at a specific temperature and thus cause reoxidation and restructuring of the oxygen-exposed surfaces, resulting in lower surface energy. This causes the anti-Ti₄O₈ crystal to nucleate and tend to grow into a nanocavity. By contrast, Ar (inert gas) does not lower the surface energy, thereby preventing nucleation and growth of anti-Ti₄O₈ crystals. In this case, the nanorods will shrink to remove unstable small empty spaces.

The compositions of the invention with nanocavities provide for an enhanced UV absorbance relative to analogous compositions without nanocavities. Accordingly, the compositions of the invention are suitable for use in UV screening compositions, such as sunscreens, cosmetics, and industrial coatings, as well as methods of enhancing the efficiency of UV absorbance of sunscreens, cosmetics, and industrial coatings. In sunscreen and cosmetic applications, UV screening compositions will prevent skin from being exposed to harmful UVA and UVB radiation. In industrial coating application, UV screening compositions will prevent discoloration due to UV radiation.

Ultraviolet-blocking compositions for sunscreen and cosmetic applications include both a metal oxide nanoparticle having regular polyhedral nanocavities and a cosmetically-acceptable carrier. Some examples of cosmetically-acceptable carriers contemplated for use in accordance with the present invention are set forth in the following patents and publications, each incorporated by reference in its entirety as if fully set forth in this specification: U.S. Pat. No. 5,700,451 (Yue et al.), U.S. Pat. No. 5,498,406 (Nearn et al.), U.S. Pat. No. 5,543,136 (Aldous), Int'l. App. No. WO90/11067 (Boots), “Scattering & Absorption of UV Radiation by Sunscreens Containing Fine Particle & Pigmentary Titanium Dioxide” Drug and Cosmetic Industry, pp. 32-39 (Robb et al., March 1994), “Testing UVA and UVB protection from Microfine Titanium Dioxide” Cosmetics and Toiletries Vol. 105, pp. 69-73 (Brown et al., December 1990). Other variations will be well-known and appreciated by persons of ordinary skill in the art.

Ultraviolet-blocking compositions for industrial coatings applications include both a metal oxide nanoparticle having regular polyhedral nanocavities and an industrially-acceptable carrier. Some examples of industrially-acceptable carriers contemplated for use in accordance with the present invention are set forth in the following patents, each incorporated by reference in its entirety as if fully set forth in this specification: U.S. Pat. No. 6,653,356 (Sherman), U.S. Pat. No. 6,074,474 (Broome et al.), U.S. Pat. No. 6,005,044 (Karl et al.), U.S. Pat. No. 5,219,918 (Guillame et al.), U.S. Pat. No. 5,112,402 (Freeman et al.), and European Patent No. EP 0 270 472 B1.

In view of the enhanced UV absorption, the compositions of the invention with nanocavities are suitable for use in photo-electrochemical systems, such as dye-sensitized TiO₂ electrodes for photovoltaics and solar cells (i.e., solar energy conversion), water splitting catalysts for hydrogen generation, and for creation or degradation of specific compounds. The compositions of the invention with nanocavities are also suitable for use in a method for enhancing the efficiency of a solar energy conversion system. Solar energy conversion systems contemplated for use in accordance with the present invention are set forth in the following publications, each incorporated by reference in its entirety as if fully set forth in this specification: K. Honda, A. Fujishima, Nature 1972, 238, 37; T. Kawai, T. Sakata, Nature 1980, 286, 31; B. O'Regan, M. Gratzel, Nature 1991, 353, 737; Z. L. Wang, Ann. Rev. Phys. Chem. 2004, 55, 159; and J. J. Park, S. Kim, A. Bard, Nano Lett. 2006, 6, 24.

It will be readily understood to one of ordinary skill in the art that the molar absorption of the compositions of the invention with nanocavities can be tuned. For example, doping of the nanoparticles may enhance the molar absorption coefficient in narrower light ranges. Suitable dopants may include lithium, sodium, potassium, boron, carbon, nitrogen, fluorine, aluminum, silicon, phosphorous, sulfur, calcium, scandium, vanadium, chromium, manganese, iron, cobalt, nickel, copper, and zinc. Preferable dopants include iron, nitrogen, and nickel. Furthermore, by modifying the size of the individual nanocavities or their size distributions, the absorbance and light wavelength range of absorption may be tuned.

The compositions of the invention with nanocavities are also usable in a method of enhancing the lithium ion storage efficiency of lithium ion batteries. Lithium ion batteries contemplated for use in accordance with the present invention are set forth in the following patents, each incorporated by reference in its entirety as if fully set forth in this specification: U.S. Pat. No. 6,749,648 (Kumar et al.); U.S. Pat. No. 6,878,490 (Gao et al.); U.S. Pat. No. 6,136,287 (Home et al.); U.S. Pat. No. 5,869,208 (Miyasaka); and U.S. Patent Application Publication No. US2006/0093913 (Howard et al.).

What follows are several non-limiting examples illustrating several embodiments of the invention.

### EXAMPLE 1

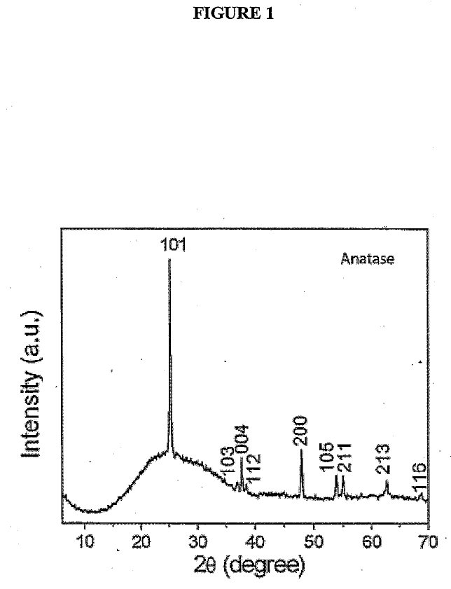

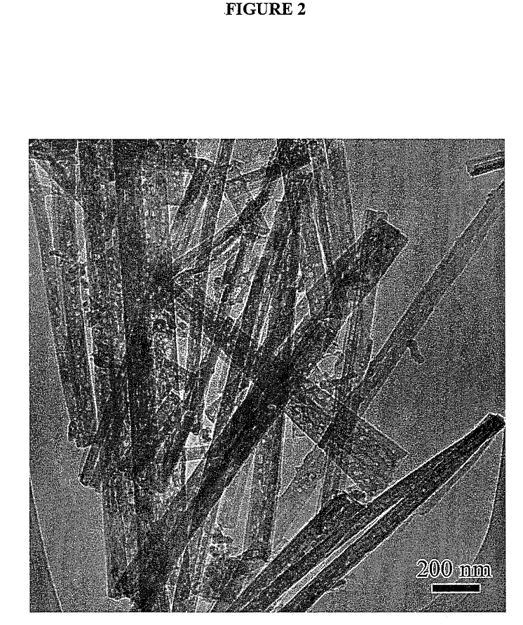

1 gram of anatase TiO₂ and 30 mL of 10N NaOH were placed in a polyfluoroethylene container. The container was places in an autoclave and heated in an oven at a temperature of 170° C. for 3 days. After this alkali treatment, the product was removed and placed in a flask containing 500 mL of a solution of hydrochloric acid (1.5% by weight) and stirred for 16 hours. The solid product was filtered from the solution and dried naturally. The dried product was then heated in air for two hours at 675° C. The resultant solid was shown to be anatase TiO₂ nanorods with regular polyhedral nanocavities by XRD as illustrated in FIG. 1, low resolution TEM as illustrated in FIG. 2, and high resolution TEM as illustrated in FIG. 2. Preliminary results indicate that the resultant solid absorbed substantially more light in the UV-region compared with analogous material without nanocavities (prepared by heat treatment under argon instead of air).

### EXAMPLE 2

Fe-doped TiO₂ with nanocavities was prepared by the following method: 1 gram of anatase TiO₂ and 30 mL of 10N NaOH were placed in an iron container. The container was placed in an autoclave and heated in an oven at a temperature of 160° C. for 3 days with a stirring speed of approximately 1000 Hz. After this alkali treatment, the product was removed and placed in a glass bottle with 500 mL of a solution of nitric acid (3% by weight) and stirred for 12 hours. The solid product was filtered from the solution and dried naturally. The dried product was heated in a pure oxygen atmosphere for two hours at 600° C.

### EXAMPLE 3

N-doped TiO₂ with nanocavities was prepared by the following method: 1 gram of anatase TiO₂ and 30 mL of 15N KOH were placed in a fluoropolymer container. The container was placed in an autoclave and heated in an oven at a temperature of 170° C. for 3 days. After this alkali treatment, the product was removed and placed in a flask with 500 mL of a solution of hydrochloric acid (1.5% by weight) and stirred for 16 hours. The solid product was filtered from the solution and dried naturally. The dried product was then heated in ammonia for two hours at 650° C.

### EXAMPLE 4

Ni-doped TiO₂ with nanocavities was prepared by the following method: 1 gram of anatase TiO₂, 0.1 gram of Ni(NO₃), and 30 mL of 10N NaOH were placed in a fluoropolymer container. The container was placed in an autoclave and heated in an oven at a temperature of 170° C. for 3 days. After this alkali treatment, the product was removed and placed in a flask with 500 mL of a solution of nitric acid (1.5% by weight) and stirred for 16 hours. The solid product was filtered from the solution and dried naturally. The dried product was heated in air for two hours at 650° C.

### EXAMPLE 5

The nanorods with nanocavities as prepared in Example 1 were studied by XRD as illustrated in FIG. 1. The XRD spectrum was collected with CuKα radiation on a Rigaku/Miniflex diffractometer. The XRD pattern shows that the nanorods with nanocavities are single-phase anatase TiO₂, in agreement with previous reports.

The nanorods with nanocavities as prepared in Example 1 were studied by low-magnification TEM as illustrated in FIG. 2. The low-magnification TEM image shows TiO₂ nanorods with a high density of nanocavities. The low-magnification TEM image also shows that the TiO₂ nanorods have a variety of sizes and that nanocavities are present in all nanorods.

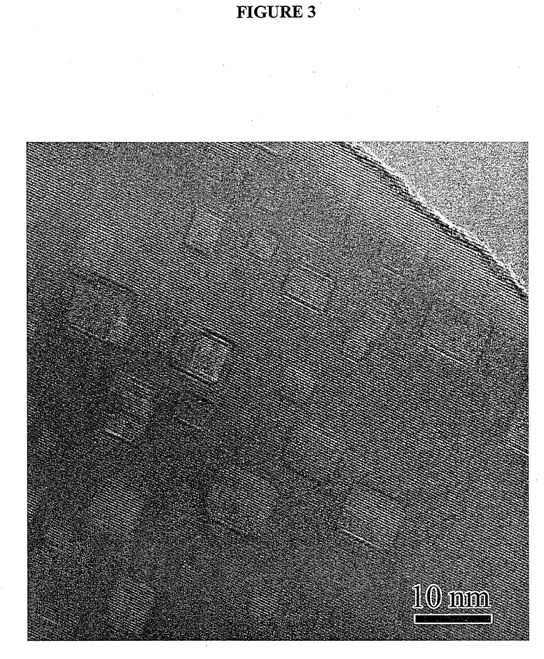

The nanorods with nanocavities as prepared in Example 1 were also studied by high-magnification TEM as illustrated in FIG. 3. The high-magnification TEM image shows a TiO₂ nanorod having a plurality of regular polyhedral nanocavities, viewed along the [100] direction.

The analytical TEM results were obtained using a JEOL 3000F transmission electron microscope, equipped with an ultra-high-resolution objective lens pole piece, an annular dark-field detector, and a post-column Gatan imaging filter. The instrument is capable of being operated in either conventional TEM or STEM modes.

It is believed that nanoparticles with nanocavities, in accordance with this invention enhance the ability of the nanoparticale to absorb light, particularly in the UV range. Further support is provided in theoretical calculations set forth in “Enhanced optical absorption in a thin silicon layer with nanovoids” (Nanotechnology, 16 (2005), 1542-48), the disclosure of which is incorporated by reference as if fully set forth in the specification.

It will be appreciated by persons skilled in the art that the present invention is not limited to what has been particularly shown and described above. It should further be understood that the above description is only representative of illustrative embodiments of the invention. The above description sets forth various embodiments which illustrate the principles of the present invention. Other embodiments may result from a different combination of portions of different embodiments.

The description has not attempted to exhaustively enumerate all possible variations. The alternate embodiments may not have been presented for a specific portion of the invention, and may result from a different combination of described portions, or that other undescribed alternate embodiments may be available for a portion, is not to be considered a disclaimer of those alternate embodiments. It will be appreciated that many of those undescribed embodiments are within the literal scope of the following claims, and others are equivalent.

1. A composition comprising a plurality of metal oxide nanoparticles having regular polyhedral nanocavities, said nanocavities being isolated from the surface of said nanoparticle

2. The composition according to claim 1, wherein the metal oxide is titania.

3. The composition according to claim 2, wherein the titania is doped with a dopant.

4. The composition according to claim 3, wherein the dopant is lithium, sodium, potassium, boron, carbon, nitrogen, fluorine, aluminum, silicon, phosphorous, sulfur, calcium, scandium, vanadium, chromium, manganese, iron, cobalt, nickel, copper, or zinc.

5. The composition according to claim 1, wherein the nanocavities have a diameter of about 5 to about 50 nanometers.

6. The composition according to claim 1, wherein the nanoparticles are nanorods, each having a diameter of about 20 to about 150 nanometers.

7. An ultraviolet-blocking composition comprising a metal oxide nanoparticle having regular polyhedral nanocavities and a cosmetically-acceptable carrier or an industrially-acceptable carrier, said nanocavities being isolated from the surface of said nanoparticle.

8. The ultraviolet-blocking composition of claim 7, wherein the ultraviolet-blocking efficiency is enhanced relative to an analogous composition without nanocavities.

9. A method of enhancing the efficiency of an ultraviolet-blocking composition comprising including in the composition the metal oxide nanoparticle having regular polyhedral nanocavities of claim 1.

10. A method of enhancing the efficiency of solar energy conversion by increasing UV absorption in a solar energy conversion system comprising including in the system the metal oxide nanoparticle having regular polyhedral nanocavities of claim 1.

11. A method of enhancing the lithium ion storage efficiency of lithium ion batteries comprising including in said batteries the metal oxide nanoparticle having regular polyhedral nanocavities of claim 1.

12. A method for producing a plurality of metal oxide nanoparticles having regular polyhedral nanocavities comprising treating a powder of the oxide with an alkali solution in an autoclave at a temperature of from about 150° C. to about 190° C., washing the alkali-treated product with an acidic solution having from about 1% to about 15% by weight of a mineral acid, and heating the washed product at a temperature of from about 550° C. to about 750° C. in an atmosphere comprising oxygen or ammonia.

13. The method of claim 12, wherein the metal oxide is anatase or rutile titania.

14. The method of claim 12, wherein the alkali solution comprises sodium hydroxide or potassium hydroxide in a concentration from about 5 molar to about 20 molar.

15. The method of claim 12, wherein the powder of the oxide is treated with an alkali solution for about 1 day to about 7 days.

16. The method of claim 12, wherein the alkali-treated product is washed with an acidic solution comprising hydrochloric acid, nitric acid, sulfuric acid, or hydrofluoric acid in a concentration from about 1% to about 15% by weight.

17. The method of claim 12, wherein the washed product is heated for about 1 to about 8 hours.

18. The method of claim 12, wherein said atmosphere further comprises argon or nitrogen.
